(19) United States
(12) Patent Application Publication
(10) Pub. No.: US 2008/0051453 A1
(43) Pub. Date: Feb. 28, 2008

(54) Sorbic acid analog co-crystals

(76) Inventors: Annette Bak, Watertown, MA (US); Drazen Ostovic, Newbury Park, CA (US)

Correspondence Address: AMGEN INC., MAIL STOP 28-2-C, ONE AMGEN CENTER DRIVE, THOUSAND OAKS, CA, 91320-1799, US

(21) Appl. No.: 11/895,071

(22) Filed: Aug. 22, 2007

Related U.S. Application Data
(60) Provisional application No. 60/839,581, filed on Aug. 22, 2006.

(51) Int. Cl. A61K 31/19; C07C 57/03; C07C 57/10

(52) U.S. Cl. 514/557; 562/598; 562/601

(57) ABSTRACT

The present invention relates to a pharmaceutical co-crystal comprising an active pharmaceutical ingredient and a co-crystal agent having the structure R¹—C(═O)XH.

This application claims the benefit of U.S. Provisional Application No. 60/839,581, filed Aug. 22, 2006, which is hereby incorporated by reference.

### BACKGROUND

Co-crystals, under names such as organic molecular compounds or complexes, have been described in the literature as far back as the 1890's, where Ling investigated halogen derivatives of quinhydrone (1). A quinohydrone may be thought of as a bulk 1:1 stoichiometric complex of hydroquinone with a quinone, held together by a network of hydrogen bonding and π-stacking. These systems are described in detail by several authors (2,3,4,5) not because of their relevance as pharmaceutical co-crystals but because of their use in photographic films. The mobility of hydroquinones themselves caused an unwanted reaction with silver halide prior to film development. This was prevented by using quinhydrone complexes that are insoluble and immobile prior to film development (5), thereby illustrating the use of co-crystals to modify the solubility of organic compounds.

Co-crystals have been widely applied in sciences other than pharmaceutical. Examples include prediction of crystal structure by using co-crystals and two dimensional laminated solids (6), and to study the separation mechanism of stationary phases and the interaction of the analyte with the column material in chiral chromatography (7).

In this application, the term “co-crystals” is meant to define crystalline phase wherein at least two components of the crystal interact by hydrogen bonding and possibly by other non-covalent interactions rather than by ion pairing. The primary difference is the physical state of the pure isolated compound. If one component is liquid at room temperature, the crystals are referred to as solvates; if both components are solids at room temperature, the products are referred to as co-crystals (8).

Co-crystals have been prepared by a variety of techniques such as melt crystallization, grinding (9) and re-crystallization from solvents (10). Co-crystals may offer an alternate approach over salt formation and formulation approaches to enhance the bioavailability of insoluble compounds (8). Like salts, co-crystals have the advantage that they can be screened for in a high-throughput platform (11). Data is also available to enable a structured search for successful co-crystals formers to compounds possessing certain functional groups. Zaworotko et al. described in a recent article use of the CSD to search for co-crystals formers for Carbamazepine (12).

Co-crystals are relatively novel in the pharmaceutical field and have not been described extensively in the literature. Most of the literature on pharmaceutical co-crystals concentrates on crystal engineering, preparation techniques, and solid-state characterization. A crystal engineering perspective is also offered in a study investigating formation of co-crystals from Ibuprofen, Flurbiprofen and Aspirin with dipyridyls as the non-pharmaceutical component. The authors conclude that the nature of the non-pharmaceutical component can dramatically affect the crystal packing and therefore also the physical properties. For example some of the co-crystals formed had higher and some lower melting points as compared to their pure components (13). Co-crystal formation of Carbamazepine has been investigated. Eight polymorphs and pseudo polymorphs for the epilepsy drug have been reported, thereby making the drug an excellent candidate for co-crystal formation. Two strategies are pursued. One strategy attempts to preserve the hydrogen bonds that exist between carboxamide groups in neighboring molecules of Carbamazepine in the crystal structure of the parent compound. Another strategy attempts to break these bonds, resulting in completely re-engineered crystals. Several multi component phases or co-crystals were formed using both strategies. Moisture was not excluded from these experiments, and therefore these phases appear to be formed in preferences over the low solubility hydrate, which is responsible for the low exposure of Carbamazepine (14).

It has been debated whether or not co-crystals can exhibit polymorphism themselves, since it is argued that a parent drug with many polymorphs will be more prone to forming co-crystals (8). In another study using solvent drop grinding, caffeine and glutaric acid, polar versus non-polar organic solvents were found to give two different polymorphs of the co-crystals (10). Finally, Zaworotko et al. made a hydrated form of carbamazepine/4-aminobenzoic acid co-crystals (12) thereby illustrating that co-crystals may be polymorphic as well as pseudopolymorphic.

Co-crystals may be used as an alternative to, or complimentary with, salt formation. However, only few examples of pharmaceutical co-crystals, where dissolution behavior is studied, have been described in the literature. One interesting example describes co-crystal formation with Fluoxetine Hydrochloride, a salt, with organic acids such as benzoic acid, fumaric acid, and succinic acids. The approach is based on halide ions as hydrogen bonding acceptors. The authors also performed powder dissolution experiments, and showed that two of the three co-crystals (fumaric acid and succinic acids co-crystals) had higher dissolution rate as compared to Fluoxetine Hydrochloride (15). In another study the formation of fumaric acid, succinic acid, and L-malic acid co-crystals of an extremely water-insoluble anti-fungal drug, itraconazole, is described. The co-crystals were reported to have similar dissolution profiles to the amorphous drug and superior to the crystalline compound thereby indicating the potential for enhanced bioavailability (16).

### REFERENCES

1. Ling, A. R. and Baker, J. K. (1893) Halogen derivatives of quinone. Part III. Derivatives of quinhydrone, J. Chem. Soc. Trans. 63, 1314-1327
2. Patil, A. O., Curtin, D. Y., and Paul, I. C. (1984) Interconversion by hydrogen transfer of unsymetrically substituted quinohydrones in the solid state. Crystal structure of the 1:2 complex of 2,5-dimethylbenzoquinone with hydroquinone, J. AM. Chem. Soc. 106, 4010-4015
3. Scheffer, J. R., Wong, Y.-F., Patil, A. O., Curtin, D. Y., and Paul, I. C. (1985) CPMAS 13C NMR Spectra of Quinones, hydroquinones, and their complexes. Use of CMR to follow a reaction in the solid state, J. AM. Chem. Soc. 107, 4898-4904
4. Patil, A. O., Curtin, D. Y., and Paul, I. C. (1984) Solid-state formation of quinhydrones from their components. Use of solid-solid reactions to prepare compounds not accessible from solution, J. AM. Chem. Soc. 106, 348-353
5. Guarrera, D., Taylor, L. D., and Warner, J. C. (1994) Molecular self-assembly in the solid-state. The combined use of solid-state NMR and differential scanning calorimetry for the determination of phase constitution, Chem. Mater. 6, 1293-1296
6. Zaworotko, M. J. (2001) Superstructural diversity in two dimensions: crystal engineering of liminate solids, Chem. Commun. 1-9
7. Koscho, M. E., Spence, P. L., and Pirkle, W. H. (2005) Chiral recognition in the solid state: crystalographically characterized diastereomeric co-crystals between a synthetic chiral selector (whelk-O1) and a representative chiral analyte, Assymmetry 16, 3147-3153
8. Almarsson, O. and Zaworotko, M. J. (2004) Crystal engineering of the composition of pharmaceutical phases. Do pharmaceutical co-crystals represent a new path to improved medicines?, Chem. Commun. 1889-1896
9. Tanaka, K. and Toda, F. (2000) Solvent-Free Organic Synthesis, Chem. Rev. 100, 1025-1074
10. Trask, A. V., Motherwell, W. D. S., and Jones, W. (2004) Solvent-drop grinding: green polymorph control of co-crystallization, Chem. Commun. 890-891
11. Morissette, S. I., Almarsson, O., Peterson, M. L., Remenar, J. F., Read, M. J., Lemmo, A. V., Ellis, S., Cima, M. J., and Gardner, C. R. (2004) High-throughput Crystallization: polymorphs, salts, co-crystals, and solvates of pharmaceutical solids, Adv. Drug Del. Rev. 56, 275-300
12. McMahon, J. A., Bis, J. A, Visweshwar, P, Shattock, T. R., McLauglin, O. L, and Zaworotko, M. J. (2005) Crystal engineering of the composition of pharmaceutical phases. 3. Primary amide supramolecular heterosynthons and their role in design of pharmaceutical co-crystals, Z. Kristallogr. 220, 340-350
13. Bailey Walsh, R. D., Bradner, M. W., Fleischman, S., Morales, L. A., Moulton, B., Rodriguez-Hornedo, N., and Zaworotko, M. J. (2003) Crystal engineering of the composition of pharmaceutical phases, Chem. Commun. 186-187
14. Fleischman, S. G., Kuduva, S. S., McMahon, J. A., Moulton, B., Bailey Walsh, R. D., Rodriguez-Hornedo, N., and Zaworotko, M. J. (2003) Crystal engineering of the composition of pharmaceutical phases: multi-component crystalline solids involving carbamazepine, Crystal Growth and Design 3, 909-919
15. Childs, S. L., Chyall, L. J., Dunlap, J. T., Smolenskaya, V. N., Stahly, B. C., and Stahly, G. P. (2004) Crystal engineering approach to forming cocrystals of amine hydrochlorides with organic acids. Molecular complexes of fluoxetine hydrochloride with benzoic, succinic, and fumaric acid, J. AM. Chem. Soc. 126, 13335-13342
16. Remenar, J. F., Morissette, S. I., Peterson, M. L., Moulton, B., MacPhee, J. M., Guzman, H. R., and Almarsson, O. (2003) Crystal engineering of novel cocrystals of a triazole drug with 1,4-dicarboxylic acids, J. AM. Chem. Soc. 125, 8456-8457

### SUMMARY

The present invention relates to a pharmaceutical co-crystal comprising an active pharmaceutical ingredient and a co-crystal agent having the structure R¹—CO₂H. The foregoing merely summarizes certain aspects of the invention and is not intended, nor should it be construed, as limiting the invention in any way. All patents, patent applications and other publications recited herein are hereby incorporated by reference in their entirety.

### DETAILED DESCRIPTION

One aspect of the current invention relates to a pharmaceutical co-crystal comprising:

an active pharmaceutical ingredient; and

a co-crystal agent having the structure R¹—C(═O)XH, wherein X is O, N(C₁₋₆alkyl) or NH and R¹ is a C₃₋₈alkyl group containing at least one trans-oriented double bond and being substituted by 0, 1, 2, 3 or 4 groups independently selected from halo, phenyl and hydroxyl.

In another embodiment, in conjunction with any of the above or below embodiments, the R¹—C(═O)XH portion of the co-crystal agent has a pKa value at least three units higher than the most basic functional group of the active pharmaceutical ingredient.

In another embodiment, in conjunction with any of the above or below embodiments, the R¹—C(═O)XH portion of the co-crystal agent has a pKa value at least four units higher than the most basic functional group of the active pharmaceutical ingredient.

In another embodiment, in conjunction with any of the above or below embodiments, the R¹—C(═O)XH portion of the co-crystal agent has a pKa value at least five units higher than the most basic functional group of the active pharmaceutical ingredient.

In another embodiment, in conjunction with any of the above or below embodiments, the R¹—C(═O)XH portion of the co-crystal agent has a pKa value at least six units higher than the most basic functional group of the active pharmaceutical ingredient.

In another embodiment, in conjunction with any of the above or below embodiments, the R¹—C(═O)XH portion of the co-crystal agent has a pKa value at least seven units higher than the most basic functional group of the active pharmaceutical ingredient.

In another embodiment, in conjunction with any of the above or below embodiments, X is O.

In another embodiment, in conjunction with any of the above or below embodiments, X is NH.

In another embodiment, in conjunction with any of the above or below embodiments, X is N(C₁₋₆alkyl).

In another embodiment, in conjunction with any of the above or below embodiments, the co-crystal agent is selected from sorbic acid, trans-2-hexenoic acid, trans-3-hexenoic acid, trans-4-hexenoic acid, trans-2-butenoic acid, trans-2-pentenoic acid, trans-3-pentenoic acid, trans-2,4-pentadienoic acid.

In another embodiment, in conjunction with any of the above or below embodiments, the co-crystal agent is selected from sorbic acid amide, trans-2-hexenoic acid amide, trans-3-hexenoic acid amide, trans-4-hexenoic acid amide, trans-2-butenoic acid amide, trans-2-pentenoic acid amide, trans-3-pentenoic acid amide, trans-2,4-pentadienoic acid amide.

In another embodiment, in conjunction with any of the above or below embodiments, the co-crystal agent is sorbic acid.

Another aspect of the invention relates to a method of manufacturing a pharmaceutical co-crystal according any of the above and below embodiments, comprising the steps of:

contacting a co-crystal agent with an active pharmaceutical ingredient;

isolating the formed pharmaceutical co-crystal.

In another embodiment, in conjunction with any of the above or below embodiments, the contacting occurs with both the co-crystal agent and the active pharmaceutical ingredient dissolved in a solvent.

In another embodiment, in conjunction with any of the above or below embodiments, the contacting occurs in a milling device with both the co-crystal agent and the active pharmaceutical ingredient being solids.

Another aspect of the invention relates to a pharmaceutical composition comprising:

a co-crystal as described above; and

a pharmaceutically-acceptable carrier or diluent.

Another aspect of the invention relates to a method for increasing the bioavailability of an active pharmaceutical ingredient in a mammal comprising the steps of contacting the active pharmaceutical ingredient with a co-crystal agent; and forming a co-crystal comprising the active pharmaceutical ingredient and the co-crystal agent.

In another embodiment, in conjunction with any of the above or below embodiments, the bioavailability is increased at least two fold.

In another embodiment, in conjunction with any of the above or below embodiments, the bioavailability is increased at least three fold.

In another embodiment, in conjunction with any of the above or below embodiments, the bioavailability is increased at least four fold.

In another embodiment, in conjunction with any of the above or below embodiments, the bioavailability is increased at least eight fold.

Examples of how to form and test co-crystals can be found in the following publications, hereby incorporated by reference in their entirety: WO 04/064762, WO 04/078161 and WO 04/078163.

Examples of active pharmaceutical ingredients include, but are not limited to, the examples and generic descriptions found in the following publications, hereby encorporated by reference in their entirety: US 20030158188, US 20030158198, US 20030158198, US 20040157845, US 20040157849, US 20040209884, US 20050009841, US 20050080095, US 20050085512, WO 02008221, WO 02030956, WO 02072536, WO 02076946, WO 02090326, WO 03006019, WO 03014064, WO 03022809, WO 03029199, WO 03049702, WO 03053945, WO 03055484, WO 03055484, WO 03055848, WO 03062209, WO 03066595, WO 03068749, WO 03070247, WO 03074520, WO 03080578, WO 03093236, WO 03095420, WO 03097586, WO 03097670, WO 03099284, WO 04002983, WO 04007459, WO 04007495, WO 04011441, WO 04014871, WO 04024710, WO 04028440, WO 04029031, WO 04029044, WO 04033435, WO 04035533, WO 04035549, WO 04046133, WO 04052845, WO 04052846, WO 04054582, WO 04055003, WO 04055004, WO 04056774, WO 04058754, WO 04072020, WO 04072069, WO 04074290, WO 04078101, WO 04078744, WO 04078749, WO 04089877, WO 04089881, WO 04096784, WO 04099177, WO 04100865, WO 04103281, WO 04108133, WO 04110986, WO 04111009, WO 05003084, WO 05004866, WO 05007646, WO 05007648, WO 05007652, WO 05009977, WO 05009980, WO 05009982, WO 05009987, WO 05009988, WO 05012287, WO 05014580, WO 05016915, WO 05016922, WO 05030753, WO 05030766, WO 05032493, WO 05033105 and WO 05035471.

Unless otherwise specified, the following definitions apply to terms found in the specification and claims:

“C_{α-β}alkyl” means an alkyl group comprising a minimum of a and a maximum of β carbon atoms in a branched, cyclical or linear relationship or any combination of the three, wherein α and β represent integers. The alkyl groups described in this section may also contain one or two double or triple bonds. Examples of C₁₋₆alkyl include, but are not limited to the following:

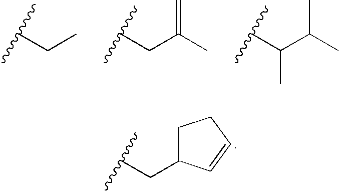

### “Halo” or “halogen” means a halogen atoms selected from F, Cl, Br and I.

It should be noted that compounds of the invention may contain groups that may exist in tautomeric forms, such as cyclic and acyclic amidine and guanidine groups, heteroatom substituted heteroaryl groups (Y′=O, S, NR), and the like, which are illustrated in the following examples:

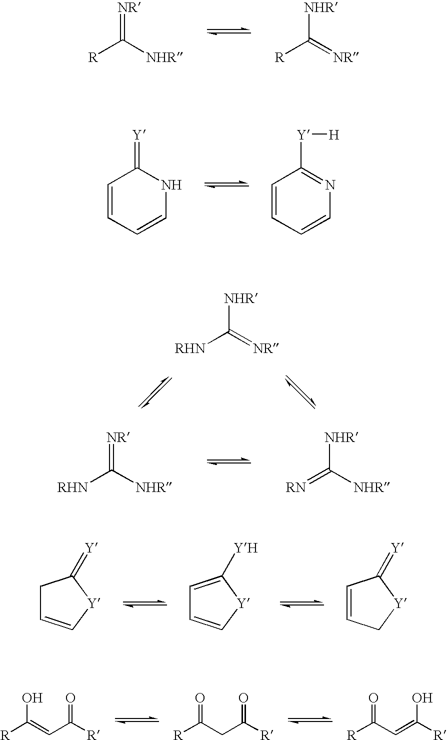

and though one form is named, described, displayed and/or claimed herein, all the tautomeric forms are intended to be inherently included in such name, description, display and/or claim.

The specification and claims contain listing of species using the language “selected from . . . and . . . ” and “is . . . or . . . ” (sometimes referred to as Markush groups). When this language is used in this application, unless otherwise stated it is meant to include the group as a whole, or any single members thereof, or any subgroups thereof. The use of this language is merely for shorthand purposes and is not meant in any way to limit the removal of individual elements or subgroups as needed.

### Experimental

Generally, co-crystals may be formed as follows:

### Materials:

1 eq drug

1.05 eq co-crystal former (for 1:1 ratio) or 2.10 eq (for 1:2 ratio)

Liquid formulation vehicle with other necessary inert excipients added such as surfactants for wetting

Slurry Method: Add co-crystal former and drug to the formulation vehicle and provide the necessary energy to mediate conversion. For some drugs, sonication with a sonicating probe will be needed. For others sonicating on a water bath or even light stirring will be sufficient. The conversion should be follow by a suitable solid-state characterization technique such as X-ray powder diffraction.

### Materials

Co-crystal formers were purchased from Sigma-Aldrich, Fluka, TCI, EM Science, Alfa Aesar and EMD Chemicals (source of sorbic acid).

### Milling

API and co-crystal former were ball milled with or without approximately 20 μL of isopropyl alcohol, acetone, methanol, ethyl acetate or 2-butanol in a mixer mill MM301 (Retsch Inc., Newton, Pa.) at a 1:1.2 ratio of API to co-crystal former in a 1.5 mL stainless steel grinding jar containing a 5 mm stainless steel grinding ball for 2 min.

### Crystallization

Crystallizations were accomplished by slow cooling a saturated solution. API and co-crystal former were dissolved in a 1:1.2 ratio in isopropyl alcohol, isopropyl acetate, acetone, methanol, ethyl acetate, dichloromethane, 1,2-dichloroethane or 2-butanol at 50° C. (or less depending on boiling point) then cooled at 2° C./min in an Imperial V oven (Lab-Line Instruments Inc., Melrose Park, Ill.). If crystallization did not occur within 48-72 hrs, slow evaporation was also utilized.

### Thermal Analysis

Differential scanning calorimetry was performed on a Q100 (TA Instruments, New Castle, Del.) at 2 or 10° C./min from 30-250° C. in an open, aluminum pan. Thermal gravimetric analysis was performed on a Q500 (TA Instruments) at 2 or 10° C./min from 30-300° C. in a platinum pan.

### X-Ray Powder Diffractometry

X-ray diffraction patterns were obtained on an X'Pert PRO x-ray diffraction system (PANalytical, Almelo, the Netherlands). Samples were scanned in continuous mode from 5-45° (2θ) step size 0.0334 on a spinning stage at 45 kV and 40 mA with CuKα radiation (1.54 Å). The incident beam path was equipped with a 0.02 rad solar slit, 15 mm mask, 4° fixed anti-scatter slit and a programmable divergence slit. The diffracted beam was equipped with a 0.02 rad solar slit, programmable anti-scatter slit and a 0.02 mm nickel filter. Detection was accomplished with an RTMS detector (X'Cellerator).

### Microscopy

Microscopy was obtained on an Eclipse E600 POL (Nikon Inc., Melville, N.Y.) equipped with an LTS 350 heating/freezing stage (Linkam Scientific Instruments Ltd., England). Samples were analyzed from 25-300° C. at 10° C./min at 100× magnification.

### NMR

¹H NMR analysis was performed on a Bruker 400 MHz NMR (Bruker BioSpin GmbH, Germany) in DMSO-d₆ or chloroform-d at 25° C.

### Hygroscopicity

Hygroscopicity was determined by dynamic vapor sorption on the DVS Advantage (Surface Measurement Systems Ltd, London). Measurements were taken from 0-90-0% RH at 25° C. with equilibration set to dm/dt+0.002%/min for 5 min or 120 min/step (min. 10 min/step).

### Solubility

Solubility was measured from a slurry (3.33 mg/mL) in FaSIF (5 mM taurocholic acid sodium and 1.5 mM lecithin in pH 6.8 phosphate buffer) with measurements taken at 1, 15, 30, 45, 60, 90, 120, 240 and 1440 min. Samples were filtered through a 0.2μ PTFE syringe filter. Analysis by HPLC-UV on an Agilent 1100 series HPLC (Agilent Technologies, Palo Alto, Calif.) equipped with a binary pump (G1312A), DAD detector (G1315B), autosampler (G1329A) and a 4.5×150 mm YMC ProC₁₈ column (Waters Corporation, Milford, Mass.). Gradient method run from 10-95% acetonitrile 0.1% triflouroacetic acid at 1 mL/min for 15 min. Standards were prepared in 50% acetonitrile at 0.05 mg/mL and injected at 1, 5, 10 and 15 μL.

### Particle Size

Particle size was determined by laser diffraction on the HELOS/BF with a CUVETTE disperser (Sympatec GmbH, Clausthal-Zellerfeld). Samples were suspended in 2% Hydroxypropyl methylcellulose 1% Tween 80 by vortex. The suspension was then added drop wise to the 50 mL cuvette containing water until a 5-15% optical concentration was achieved. Measurements were taken for 10 s on the R3 or R5 lens with mixing at 500 rpm.

### Elemental Analysis

Elemental analysis was performed at Galbraith Laboratories (Knoxville, Tenn.).

### Single Crystal Structures

### EXAMPLES 1-3

Single crystal structures for N-(4-(6-(4-(trifluoromethyl)phenyl)pyrimidin-4-yloxy)benzo[d]thiazol-2-yl)acetamide trans-cinnamic acid co-crystal (Example 1) and N-(4-(6-(4-(trifluoromethyl)phenyl)pyrimidin-4-yloxy)benzo[d]thiazol-2-yl)acetamide trans-2-hexanoic acid co-crystal (Example 2) were determined as follows for Example 3:

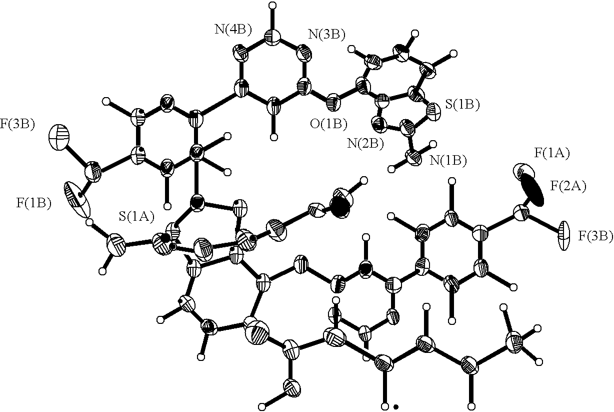

4-(6-(4-(Trifluoromethyl)phenyl)pyrimidin-4-yloxy)benzo[d]thiazol-2-amine sorbic acid co-crystal (Example 3): The colorless block crystal with dimensions 0.20×0.18×0.18 mm was mounted on a glass fiber using very small amount of paratone oil. Data were collected using a Bruker SMART CCD (charge coupled device) based diffractometer equipped with an Oxford Cryostream low-temperature apparatus operating at 193 K. A suitable crystal was chosen and mounted on a glass fiber using grease. Data were measured using omega scans of 0.3° per frame for 30 seconds, such that a hemisphere was collected. A total of 1850 frames were collected with a maximum resolution of 0.76 Å. The first 50 frames were recollected at the end of data collection to monitor for decay. Cell parameters were retrieved using SMART software and refined using SAINT on all observed reflections (SMART V 5.625 (NT) Software for the CCD Detector System; Bruker Analytical X-ray Systems, Madison, Wis. (2001)). Data reduction was performed using the SAINT software (SAINT V 6.22 (NT) Software for the CCD Detector System Bruker Analytical X-ray Systems, Madison, Wis. (2001)) which corrects for Lp and decay. Absorption corrections were applied using SADABS (Program for absorption corrections using Siemens CCD based on the method of Robert Blessing; Blessing, R. H. Acta Cryst. A51 1995, 33-38) multiscan technique, supplied by George Sheldrick. The structures are solved by the direct method using the SHELXS-97 (Sheldrick, G. M. SHELXS-90, Program for the Solution of Crystal Structure, University of Göttingen, Germany, 1990) program and refined by least squares method on F², SHELXL-97 (Sheldrick, G. M. SHELXL-97, Program for the Refinement of Crystal Structure, University of Göttingen, Germany, 1997), incorporated in SHELXTL-PC V 6.10 (SHELXTL 6.1 (PC-Version), Program library for Structure Solution and Molecular Graphics; Bruker Analytical X-ray Systems, Madison, Wis. (2000)). The structure was solved in the space group P 1̅ (# 2). All non-hydrogen atoms are refined anisotropically. Hydrogens were found by difference Fourier methods and refined isotropically. The crystal used for the diffraction study showed no decomposition during data collection. All drawing are done at 50% ellipsoids.

TABLE 1
Crystal data and structure refinement for Example 3.

| Empirical formula | C24H19F3N4O3S | |
|---|---|---|
| Formula weight | 500.49 | |
| Temperature | 193(2) K | |
| Wavelength | 0.71073 Å | |
| Crystal system | Triclinic | |
| Space group | P-1 | |
| Unit cell dimensions | a = 11.917(3) Å | α = 83.330(4)°. |
| | b = 12.426(3) Å | β = 76.227(4)°. |
| | c = 16.430(4) Å | γ = 78.659(4)°. |

| | |
|---|---|
| Volume | 2310.8(11) Å³ |
| Z | 4 |
| Density (calculated) | 1.439 Mg/m³ |
| Absorption coefficient | 0.199 mm⁻¹ |
| F(000) | 1032 |
| Crystal size | 0.20 × 0.18 × 0.16 mm³ |
| Theta range for data collection | 1.28 to 27.91°. |
| Index ranges | −15 <= h <= 15, −16 <= k <= 16, |
| | −21 <= 1 <= 21 |
| Reflections collected | 23122 |
| Independent reflections | 10949 [R(int) = 0.0194] |
| Completeness to theta = 27.91° | 98.7% |
| Absorption correction | Empirical |
| Max. and min. transmission | 0.9688 and 0.9613 |
| Refinement method | Full-matrix least-squares on F² |
| Data/restraints/parameters | 10949/0/783 |
| Goodness-of-fit on F² | 1.014 |
| Final R indices [I > 2sigma(I)] | R1 = 0.0463, wR2 = 0.1238 |
| R indices (all data) | R1 = 0.0585, wR2 = 0.1341 |
| Largest diff. peak and hole | 0.819 and −0.429 e.Å⁻³ |

TABLE 2
Atomic coordinates (×10⁴) and equivalent isotropic displacement
parameters (Å² × 10³) for Example 3. U(eq) is defined
as one third of the trace of the orthogonalized U^{ij} tensor.

| | x | y | z | U(eq) |
|---|---|---|---|---|
| S(1A) | −11(1) | 973(1) | 11195(1) | 37(1) |
| F(1A) | 2748(1) | 1033(1) | 2647(1) | 68(1) |
| F(2A) | 4417(2) | 1463(1) | 2568(1) | 72(1) |
| F(3A) | 4287(2) | −182(1) | 2425(1) | 71(1) |
| O(1A) | 2709(1) | 870(1) | 8402(1) | 34(1) |
| N(1A) | −1490(1) | 1908(1) | 10205(1) | 37(1) |
| N(2A) | 486(1) | 1334(1) | 9563(1) | 30(1) |
| N(3A) | 2744(1) | −926(1) | 8129(1) | 33(1) |
| N(4A) | 2988(1) | −1367(1) | 6714(1) | 37(1) |
| C(1A) | −386(2) | 1446(1) | 10227(1) | 32(1) |
| C(2A) | 1426(2) | 528(1) | 10661(1) | 34(1) |
| C(3A) | 1511(2) | 809(1) | 9795(1) | 30(1) |
| C(4A) | 2598(2) | 521(1) | 9257(1) | 32(1) |
| C(5A) | 3554(2) | −30(2) | 9563(1) | 41(1) |
| C(6A) | 3433(2) | −308(2) | 10424(1) | 44(1) |
| C(7A) | 2369(2) | −31(2) | 10978(1) | 41(1) |
| C(8A) | 2811(1) | 105(1) | 7850(1) | 29(1) |
| C(9A) | 2980(2) | 470(1) | 7005(1) | 31(1) |
| C(10A) | 3070(1) | −301(1) | 6445(1) | 29(1) |
| C(11A) | 2834(2) | −1605(2) | 7531(1) | 39(1) |
| C(12A) | 3265(1) | −36(1) | 5523(1) | 29(1) |
| C(13A) | 3619(2) | 942(1) | 5156(1) | 37(1) |
| C(14A) | 3797(2) | 1173(2) | 4291(1) | 39(1) |
| C(15A) | 3628(2) | 428(1) | 3793(1) | 34(1) |
| C(16A) | 3287(2) | −558(2) | 4151(1) | 40(1) |
| C(17A) | 3111(2) | −788(2) | 5011(1) | 38(1) |
| C(18A) | 3766(2) | 687(2) | 2865(1) | 41(1) |
| O(2A) | 8464(1) | 2618(1) | 8468(1) | 49(1) |
| O(3A) | 10249(1) | 1703(1) | 7941(1) | 45(1) |
| C(19A) | 9199(2) | 2233(2) | 7874(1) | 37(1) |
| C(20A) | 8967(2) | 2321(2) | 7029(1) | 43(1) |
| C(21A) | 9718(2) | 1867(2) | 6368(1) | 41(1) |
| C(22A) | 9484(2) | 1921(2) | 5538(1) | 43(1) |
| C(23A) | 10232(2) | 1476(2) | 4884(1) | 51(1) |
| C(24A) | 10024(3) | 1515(3) | 4022(2) | 67(1) |
| S(1B) | 5177(1) | 4118(1) | 3767(1) | 37(1) |
| F(1B) | 2628(2) | 3733(2) | 11846(1) | 119(1) |
| F(2B) | 1065(2) | 3134(2) | 12067(1) | 103(1) |
| F(3B) | 989(2) | 4799(1) | 12165(1) | 88(1) |
| O(1B) | 2042(1) | 3471(1) | 6270(1) | 35(1) |
| N(1B) | 6425(1) | 3607(1) | 4978(1) | 36(1) |
| N(2B) | 4417(1) | 3555(1) | 5351(1) | 31(1) |
| N(3B) | 2014(1) | 5293(1) | 6470(1) | 35(1) |
| N(4B) | 1826(1) | 5827(1) | 7852(1) | 37(1) |
| C(1B) | 5369(2) | 3726(1) | 4793(1) | 32(1) |
| C(2B) | 3681(2) | 4096(1) | 4124(1) | 34(1) |
| C(3B) | 3454(2) | 3782(1) | 4986(1) | 31(1) |
| C(4B) | 2293(2) | 3770(1) | 5402(1) | 34(1) |
| C(5B) | 1404(2) | 4008(2) | 4973(1) | 43(1) |
| C(6B) | 1666(2) | 4291(2) | 4111(1) | 49(1) |
| C(7B) | 2799(2) | 4359(2) | 3679(1) | 44(1) |
| C(8B) | 1956(1) | 4273(1) | 6790(1) | 31(1) |
| C(9B) | 1812(2) | 3964(1) | 7643(1) | 33(1) |
| C(10B) | 1770(2) | 4774(1) | 8163(1) | 31(1) |
| C(11B) | 1931(2) | 6017(2) | 7036(1) | 39(1) |
| C(12B) | 1685(2) | 4558(1) | 9081(1) | 31(1) |
| C(13B) | 1912(2) | 3491(2) | 9445(1) | 37(1) |
| C(14B) | 1860(2) | 3308(2) | 10298(1) | 40(1) |
| C(15B) | 1573(2) | 4191(2) | 10794(1) | 36(1) |
| C(16B) | 1332(2) | 5255(2) | 10446(1) | 39(1) |
| C(17B) | 1388(2) | 5438(1) | 9590(1) | 36(1) |
| C(18B) | 1579(2) | 3982(2) | 11709(1) | 47(1) |
| O(2B) | 6096(1) | 3262(1) | 6826(1) | 45(1) |
| O(3B) | 4372(1) | 2737(1) | 6943(1) | 44(1) |
| C(19B) | 5219(2) | 2958(2) | 7258(1) | 37(1) |
| C(20B) | 4964(2) | 2813(2) | 8181(1) | 44(1) |
| C(21B) | 5563(2) | 3195(2) | 8636(1) | 41(1) |
| C(22B) | 5299(2) | 3154(2) | 9547(1) | 47(1) |
| C(23B) | 5858(2) | 3606(2) | 9983(1) | 50(1) |
| C(24B) | 5608(3) | 3610(3) | 10915(2) | 63(1) |

TABLE 3
Bond lengths [Å] and angles [°] for Example 3.

| | | | |
|---|---|---|---|
| S(1A)—C(2A) | 1.7417(19) | S(1A)—C(1A) | 1.7586(17) |
| F(1A)—C(18A) | 1.323(2) | F(2A)—C(18A) | 1.332(2) |
| F(3A)—C(18A) | 1.333(2) | O(1A)—C(8A) | 1.3568(19) |
| O(1A)—C(4A) | 1.4048(19) | N(1A)—C(1A) | 1.335(2) |
| N(1A)—H(2A) | 0.86(2) | N(1A)—H(1A) | 0.93(2) |
| N(2A)—C(1A) | 1.315(2) | N(2A)—C(3A) | 1.383(2) |
| N(3A)—C(8A) | 1.320(2) | N(3A)—C(11A) | 1.340(2) |
| N(4A)—C(11A) | 1.317(2) | N(4A)—C(10A) | 1.360(2) |
| C(2A)—C(7A) | 1.379(3) | C(2A)—C(3A) | 1.409(2) |
| C(3A)—C(4A) | 1.391(2) | C(4A)—C(5A) | 1.380(3) |
| C(5A)—C(6A) | 1.396(3) | C(5A)—H(3A) | 0.95(2) |
| C(6A)—C(7A) | 1.381(3) | C(6A)—H(4A) | 0.97(2) |
| C(7A)—H(5A) | 0.93(2) | C(8A)—C(9A) | 1.390(2) |
| C(9A)—C(10A) | 1.376(2) | C(9A)—H(6A) | 0.97(2) |
| C(10A)—C(12A) | 1.485(2) | C(11A)—H(7A) | 0.96(2) |
| C(12A)—C(13A) | 1.388(2) | C(12A)—C(17A) | 1.393(2) |
| C(13A)—C(14A) | 1.390(2) | C(13A)—H(8A) | 0.93(2) |
| C(14A)—C(15A) | 1.377(2) | C(14A)—H(9A) | 0.95(2) |
| C(15A)—C(16A) | 1.388(3) | C(15A)—C(18A) | 1.497(2) |
| C(16A)—C(17A) | 1.382(2) | C(16A)—H(10A) | 0.97(3) |
| C(17A)—H(11A) | 0.99(2) | O(2A)—C(19A) | 1.220(2) |
| O(3A)—C(19A) | 1.318(2) | O(3A)—H(12A) | 0.87(3) |
| C(19A)—C(20A) | 1.466(3) | C(20A)—C(21A) | 1.334(3) |
| C(20A)—H(13A) | 0.91(2) | C(21A)—C(22A) | 1.447(3) |
| C(21A)—H(14A) | 0.96(2) | C(22A)—C(23A) | 1.321(3) |
| C(22A)—H(15A) | 0.98(3) | C(23A)—C(24A) | 1.488(3) |
| C(23A)—H(16A) | 0.93(3) | C(24A)—H(18A) | 0.93(4) |
| C(24A)—H(19A) | 0.92(4) | C(24A)—H(17A) | 0.94(5) |
| S(1B)—C(2B) | 1.7437(19) | S(1B)—C(1B) | 1.7551(17) |
| F(1B)—C(18B) | 1.294(3) | F(2B)—C(18B) | 1.330(3) |
| F(3B)—C(18B) | 1.316(3) | O(1B)—C(8B) | 1.3603(19) |
| O(1B)—C(4B) | 1.4066(19) | N(1B)—C(1B) | 1.341(2) |
| N(1B)—H(1B) | 0.86(2) | N(1B)—H(2B) | 0.91(2) |
| N(2B)—C(1B) | 1.313(2) | N(2B)—C(3B) | 1.385(2) |
| N(3B)—C(8B) | 1.323(2) | N(3B)—C(11B) | 1.341(2) |
| N(4B)—C(11B) | 1.315(2) | N(4B)—C(10B) | 1.357(2) |
| C(2B)—C(7B) | 1.386(3) | C(2B)—C(3B) | 1.402(2) |
| C(3B)—C(4B) | 1.391(3) | C(4B)—C(5B) | 1.375(3) |
| C(5B)—C(6B) | 1.395(3) | C(5B)—H(3B) | 0.98(2) |
| C(6B)—C(7B) | 1.380(3) | C(6B)—H(4B) | 0.93(3) |
| C(7B)—H(5B) | 0.97(2) | C(8B)—C(9B) | 1.388(2) |
| C(9B)—C(10B) | 1.380(2) | C(9B)—H(6B) | 0.95(2) |
| C(10B)—C(12B) | 1.484(2) | C(11B)—H(7B) | 0.95(2) |
| C(12B)—C(17B) | 1.392(2) | C(12B)—C(13B) | 1.394(2) |
| C(13B)—C(14B) | 1.382(2) | C(13B)—H(8B) | 0.95(2) |
| C(14B)—C(15B) | 1.384(3) | C(14B)—H(9B) | 0.99(3) |
| C(15B)—C(16B) | 1.383(3) | C(15B)—C(18B) | 1.496(2) |
| C(16B)—C(17B) | 1.385(2) | C(16B)—H(10B) | 0.93(3) |
| C(17B)—H(11B) | 0.97(2) | O(2B)—C(19B) | 1.216(2) |
| O(3B)—C(19B) | 1.324(2) | O(3B)—H(12B) | 0.92(3) |
| C(19B)—C(20B) | 1.470(3) | C(20B)—C(21B) | 1.329(3) |
| C(20B)—H(13B) | 0.95(3) | C(21B)—C(22B) | 1.451(3) |
| C(21B)—H(14B) | 0.98(2) | C(22B)—C(23B) | 1.319(3) |
| C(22B)—H(15B) | 0.94(2) | C(23B)—C(24B) | 1.490(3) |
| C(23B)—H(16B) | 1.01(3) | C(24B)—H(18B) | 0.95(4) |
| C(24B)—H(19B) | 0.95(4) | C(24B)—H(17B) | 0.93(4) |
| C(2A)—S(1A)—C(1A) | 89.28(8) | C(8A)—O(1A)—C(4A) | 118.32(12) |
| C(1A)—N(1A)—H(2A) | 117.3(16) | C(1A)—N(1A)—H(1A) | 115.4(15) |
| H(2A)—N(1A)—H(1A) | 119(2) | C(1A)—N(2A)—C(3A) | 110.28(14) |
| C(8A)—N(3A)—C(11A) | 114.74(14) | C(11A)—N(4A)—C(10A) | 115.95(14) |
| N(2A)—C(1A)—N(1A) | 124.43(16) | N(2A)—C(1A)—S(1A) | 115.49(14) |
| N(1A)—C(1A)—S(1A) | 120.06(12) | C(7A)—C(2A)—C(3A) | 122.30(16) |
| C(7A)—C(2A)—S(1A) | 128.93(13) | C(3A)—C(2A)—S(1A) | 108.76(13) |
| N(2A)—C(3A)—C(4A) | 126.27(14) | N(2A)—C(3A)—C(2A) | 116.12(15) |
| C(4A)—C(3A)—C(2A) | 117.60(16) | C(5A)—C(4A)—C(3A) | 120.95(16) |
| C(5A)—C(4A)—O(1A) | 121.08(15) | C(3A)—C(4A)—O(1A) | 117.86(15) |
| C(4A)—C(5A)—C(6A) | 119.82(18) | C(4A)—C(5A)—H(3A) | 118.4(12) |
| C(6A)—C(5A)—H(3A) | 121.7(12) | C(7A)—C(6A)—C(5A) | 120.91(19) |
| C(7A)—C(6A)—H(4A) | 122.0(13) | C(5A)—C(6A)—H(4A) | 117.0(13) |
| C(2A)—C(7A)—C(6A) | 118.42(17) | C(2A)—C(7A)—H(5A) | 120.2(15) |
| C(6A)—C(7A)—H(5A) | 121.3(15) | N(3A)—C(8A)—O(1A) | 119.84(14) |
| N(3A)—C(8A)—C(9A) | 123.47(14) | O(1A)—C(8A)—C(9A) | 116.69(14) |
| C(10A)—C(9A)—C(8A) | 116.80(14) | C(10A)—C(9A)—H(6A) | 124.9(12) |
| C(8A)—C(9A)—H(6A) | 118.3(12) | N(4A)—C(10A)—C(9A) | 121.12(14) |
| N(4A)—C(10A)—C(12A) | 116.02(13) | C(9A)—C(10A)—C(12A) | 122.86(14) |
| N(4A)—C(11A)—N(3A) | 127.90(16) | N(4A)—C(11A)—H(7A) | 116.6(12) |
| N(3A)—C(11A)—H(7A) | 115.5(12) | C(13A)—C(12A)—C(17A) | 119.00(15) |
| C(13A)—C(12A)—C(10A) | 121.42(14) | C(17A)—C(12A)—C(10A) | 119.57(14) |
| C(12A)—C(13A)—C(14A) | 120.41(16) | C(12A)—C(13A)—H(8A) | 121.5(13) |
| C(14A)—C(13A)—H(8A) | 118.0(13) | C(15A)—C(14A)—C(13A) | 120.00(16) |
| C(15A)—C(14A)—H(9A) | 120.1(14) | C(13A)—C(14A)—H(9A) | 119.9(14) |
| C(14A)—C(15A)—C(16A) | 120.20(16) | C(14A)—C(15A)—C(18A) | 120.59(16) |
| C(16A)—C(15A)—C(18A) | 119.17(16) | C(17A)—C(16A)—C(15A) | 119.78(16) |
| C(17A)—C(16A)—H(10A) | 118.5(14) | C(15A)—C(16A)—H(10A) | 121.7(14) |
| C(16A)—C(17A)—C(12A) | 120.59(16) | C(16A)—C(17A)—H(11A) | 120.5(13) |
| C(12A)—C(17A)—H(11A) | 118.9(13) | F(1A)—C(18A)—F(2A) | 106.67(17) |
| F(1A)—C(18A)—F(3A) | 105.97(17) | F(2A)—C(18A)—F(3A) | 105.71(16) |
| F(1A)—C(18A)—C(15A) | 112.47(15) | F(2A)—C(18A)—C(15A) | 112.74(16) |
| F(3A)—C(18A)—C(15A) | 112.74(16) | C(19A)—O(3A)—H(12A) | 110.3(18) |
| O(2A)—C(19A)—O(3A) | 122.89(17) | O(2A)—C(19A)—C(20A) | 121.92(18) |
| O(3A)—C(19A)—C(20A) | 115.19(16) | C(21A)—C(20A)—C(19A) | 124.21(19) |
| C(21A)—C(20A)—H(13A) | 122.1(15) | C(19A)—C(20A)—H(13A) | 113.6(15) |
| C(20A)—C(21A)—C(22A) | 124.69(19) | C(20A)—C(21A)—H(14A) | 118.0(14) |
| C(22A)—C(21A)—H(14A) | 117.3(14) | C(23A)—C(22A)—C(21A) | 124.6(2) |
| C(23A)—C(22A)—H(15A) | 118.0(14) | C(21A)—C(22A)—H(15A) | 117.4(14) |
| C(22A)—C(23A)—C(24A) | 126.1(2) | C(22A)—C(23A)—H(16A) | 116.2(17) |
| C(24A)—C(23A)—H(16A) | 117.7(17) | C(23A)—C(24A)—H(18A) | 107(2) |
| C(23A)—C(24A)—H(19A) | 109(2) | H(18A)—C(24A)—H(19A) | 104(3) |
| C(23A)—C(24A)—H(17A) | 116(3) | H(18A)—C(24A)—H(17A) | 113(3) |
| H(19A)—C(24A)—H(17A) | 107(3) | C(2B)—S(1B)—C(1B) | 89.23(8) |
| C(8B)—O(1B)—C(4B) | 116.71(12) | C(1B)—N(1B)—H(1B) | 115.0(14) |
| C(1B)—N(1B)—H(2B) | 118.4(14) | H(1B)—N(1B)—H(2B) | 118(2) |
| C(1B)—N(2B)—C(3B) | 110.44(14) | C(8B)—N(3B)—C(11B) | 114.68(14) |
| C(11B)—N(4B)—C(10B) | 116.00(14) | N(2B)—C(1B)—N(1B) | 123.36(15) |
| N(2B)—C(1B)—S(1B) | 115.43(13) | N(1B)—C(1B)—S(1B) | 121.21(13) |
| C(7B)—C(2B)—C(3B) | 122.18(17) | C(7B)—C(2B)—S(1B) | 128.91(14) |
| C(3B)—C(2B)—S(1B) | 108.89(13) | N(2B)—C(3B)—C(4B) | 126.14(15) |
| N(2B)—C(3B)—C(2B) | 115.95(15) | C(4B)—C(3B)—C(2B) | 117.85(16) |
| C(5B)—C(4B)—C(3B) | 121.09(16) | C(5B)—C(4B)—O(1B) | 119.96(16) |
| C(3B)—C(4B)—O(1B) | 118.91(15) | C(4B)—C(5B)—C(6B) | 119.44(19) |
| C(4B)—C(5B)—H(3B) | 119.2(14) | C(6B)—C(5B)—H(3B) | 121.3(14) |
| C(7B)—C(6B)—C(5B) | 121.42(19) | C(7B)—C(6B)—H(4B) | 120.5(15) |
| C(5B)—C(6B)—H(4B) | 118.1(15) | C(6B)—C(7B)—C(2B) | 117.94(17) |
| C(6B)—C(7B)—H(5B) | 124.8(13) | C(2B)—C(7B)—H(5B) | 117.3(13) |
| N(3B)—C(8B)—O(1B) | 119.49(14) | N(3B)—C(8B)—C(9B) | 123.38(15) |
| O(1B)—C(8B)—C(9B) | 117.13(14) | C(10B)—C(9B)—C(8B) | 116.75(15) |
| C(10B)—C(9B)—H(6B) | 122.9(12) | C(8B)—C(9B)—H(6B) | 120.4(12) |
| N(4B)—C(10B)—C(9B) | 121.14(15) | N(4B)—C(10B)—C(12B) | 115.76(14) |
| C(9B)—C(10B)—C(12B) | 123.09(15) | N(4B)—C(11B)—N(3B) | 127.99(16) |
| N(4B)—C(11B)—H(7B) | 114.3(13) | N(3B)—C(11B)—H(7B) | 117.7(13) |
| C(17B)—C(12B)—C(13B) | 119.10(15) | C(17B)—C(12B)—C(10B) | 119.59(15) |
| C(13B)—C(12B)—C(10B) | 121.30(14) | C(14B)—C(13B)—C(12B) | 120.51(16) |
| C(14B)—C(13B)—H(8B) | 119.5(13) | C(12B)—C(13B)—H(8B) | 119.9(13) |
| C(13B)—C(14B)—C(15B) | 119.68(17) | C(13B)—C(14B)—H(9B) | 120.8(15) |
| C(15B)—C(14B)—H(9B) | 119.5(15) | C(16B)—C(15B)—C(14B) | 120.62(16) |
| C(16B)—C(15B)—C(18B) | 120.37(17) | C(14B)—C(15B)—C(18B) | 118.96(17) |
| C(15B)—C(16B)—C(17B) | 119.62(16) | C(15B)—C(16B)—H(10B) | 121.2(15) |
| C(17B)—C(16B)—H(10B) | 119.1(15) | C(16B)—C(17B)—C(12B) | 120.46(17) |
| C(16B)—C(17B)—H(11B) | 118.4(12) | C(12B)—C(17B)—H(11B) | 121.1(12) |
| F(1B)—C(18B)—F(3B) | 108.7(2) | F(1B)—C(18B)—F(2B) | 104.7(2) |
| F(3B)—C(18B)—F(2B) | 103.40(19) | F(1B)—C(18B)—C(15B) | 112.74(17) |
| F(3B)—C(18B)—C(15B) | 113.97(18) | F(2B)—C(18B)—C(15B) | 112.55(17) |
| C(19B)—O(3B)—H(12B) | 110.7(19) | O(2B)—C(19B)—O(3B) | 123.14(16) |
| O(2B)—C(19B)—C(20B) | 124.47(17) | O(3B)—C(19B)—C(20B) | 112.38(16) |
| C(21B)—C(20B)—C(19B) | 122.49(18) | C(21B)—C(20B)—H(13B) | 120.3(15) |
| C(19B)—C(20B)—H(13B) | 117.1(15) | C(20B)—C(21B)—C(22B) | 125.14(19) |
| C(20B)—C(21B)—H(14B) | 118.3(13) | C(22B)—C(21B)—H(14B) | 116.6(14) |
| C(23B)—C(22B)—C(21B) | 123.9(2) | C(23B)—C(22B)—H(15B) | 119.6(15) |
| C(21B)—C(22B)—H(15B) | 116.5(15) | C(22B)—C(23B)—C(24B) | 126.2(2) |
| C(22B)—C(23B)—H(16B) | 118.3(16) | C(24B)—C(23B)—H(16B) | 115.4(17) |
| C(23B)—C(24B)—H(18B) | 116(2) | C(23B)—C(24B)—H(19B) | 115(2) |
| H(18B)—C(24B)—H(19B) | 102(3) | C(23B)—C(24B)—H(17B) | 109(2) |
| H(18B)—C(24B)—H(17B) | 104(3) | H(19B)—C(24B)—H(17B) | 110(3) |

TABLE 4
Anisotropic displacement parameters (Å² × 10³) for Example 3. The
anisotropic displacement factor exponent takes the form:
−2π²[h² a*²U¹¹ + . . . + 2 h k a* b* U¹²]

| | U¹¹ | U²² | U³³ | U²³ | U¹³ | U¹² |
|---|---|---|---|---|---|---|
| S(1A) | 48(1) | 40(1) | 23(1) | −5(1) | −3(1) | −14(1) |
| F(1A) | 56(1) | 104(1) | 39(1) | 1(1) | −19(1) | 2(1) |
| F(2A) | 98(1) | 91(1) | 34(1) | 14(1) | −11(1) | −49(1) |
| F(3A) | 103(1) | 64(1) | 31(1) | −13(1) | −4(1) | 12(1) |
| O(1A) | 47(1) | 32(1) | 24(1) | −5(1) | −3(1) | −11(1) |
| N(1A) | 41(1) | 34(1) | 33(1) | −4(1) | −1(1) | −8(1) |
| N(2A) | 40(1) | 27(1) | 26(1) | −4(1) | −5(1) | −10(1) |
| N(3A) | 42(1) | 32(1) | 27(1) | −1(1) | −6(1) | −12(1) |
| N(4A) | 55(1) | 29(1) | 29(1) | −2(1) | −10(1) | −12(1) |
| C(1A) | 46(1) | 26(1) | 26(1) | −4(1) | −4(1) | −13(1) |
| C(2A) | 46(1) | 33(1) | 26(1) | −6(1) | −6(1) | −15(1) |
| C(3A) | 43(1) | 28(1) | 24(1) | −5(1) | −7(1) | −13(1) |
| C(4A) | 42(1) | 31(1) | 26(1) | −5(1) | −7(1) | −12(1) |
| C(5A) | 42(1) | 44(1) | 38(1) | −7(1) | −9(1) | −12(1) |
| C(6A) | 47(1) | 50(1) | 42(1) | −4(1) | −20(1) | −9(1) |
| C(7A) | 56(1) | 46(1) | 28(1) | −2(1) | −15(1) | −18(1) |
| C(8A) | 28(1) | 31(1) | 28(1) | −6(1) | −3(1) | −6(1) |
| C(9A) | 36(1) | 27(1) | 28(1) | −3(1) | −3(1) | −7(1) |
| C(10A) | 31(1) | 27(1) | 27(1) | −2(1) | −5(1) | −5(1) |
| C(11A) | 60(1) | 29(1) | 32(1) | 0(1) | −11(1) | −14(1) |
| C(12A) | 33(1) | 27(1) | 26(1) | −4(1) | −5(1) | −3(1) |
| C(13A) | 52(1) | 30(1) | 29(1) | −4(1) | −7(1) | −11(1) |
| C(14A) | 56(1) | 30(1) | 30(1) | 0(1) | −6(1) | −11(1) |
| C(15A) | 36(1) | 37(1) | 26(1) | −3(1) | −6(1) | −2(1) |
| C(16A) | 53(1) | 39(1) | 31(1) | −8(1) | −7(1) | −12(1) |
| C(17A) | 52(1) | 31(1) | 31(1) | −4(1) | −7(1) | −13(1) |
| C(18A) | 45(1) | 47(1) | 29(1) | −3(1) | −7(1) | −4(1) |
| O(2A) | 47(1) | 58(1) | 39(1) | −10(1) | −8(1) | 2(1) |
| O(3A) | 43(1) | 58(1) | 31(1) | −2(1) | −8(1) | 0(1) |
| C(19A) | 39(1) | 37(1) | 36(1) | −2(1) | −8(1) | −9(1) |
| C(20A) | 39(1) | 50(1) | 39(1) | −2(1) | −11(1) | −4(1) |
| C(21A) | 39(1) | 46(1) | 37(1) | 1(1) | −11(1) | −7(1) |
| C(22A) | 39(1) | 54(1) | 38(1) | 1(1) | −11(1) | −7(1) |
| C(23A) | 46(1) | 64(1) | 39(1) | 1(1) | −11(1) | −3(1) |
| C(24A) | 68(2) | 91(2) | 38(1) | −3(1) | −10(1) | −8(2) |
| S(1B) | 49(1) | 37(1) | 25(1) | −2(1) | −7(1) | −7(1) |
| F(1B) | 65(1) | 242(3) | 47(1) | −27(1) | −26(1) | 5(1) |
| F(2B) | 174(2) | 108(1) | 40(1) | 24(1) | −30(1) | −67(1) |
| F(3B) | 134(2) | 85(1) | 31(1) | −14(1) | −15(1) | 16(1) |
| O(1B) | 49(1) | 32(1) | 27(1) | −5(1) | −9(1) | −13(1) |
| N(1B) | 43(1) | 33(1) | 31(1) | 0(1) | −9(1) | −8(1) |
| N(2B) | 43(1) | 27(1) | 26(1) | −2(1) | −11(1) | −8(1) |
| N(3B) | 46(1) | 31(1) | 28(1) | −1(1) | −8(1) | −8(1) |
| N(4B) | 53(1) | 28(1) | 29(1) | −2(1) | −8(1) | −9(1) |
| C(1B) | 46(1) | 23(1) | 27(1) | −3(1) | −10(1) | −5(1) |
| C(2B) | 48(1) | 31(1) | 27(1) | −5(1) | −10(1) | −8(1) |
| C(3B) | 45(1) | 25(1) | 28(1) | −5(1) | −12(1) | −8(1) |
| C(4B) | 47(1) | 31(1) | 27(1) | −6(1) | −11(1) | −10(1) |
| C(5B) | 48(1) | 46(1) | 40(1) | −7(1) | −15(1) | −10(1) |
| C(6B) | 55(1) | 59(1) | 38(1) | −5(1) | −24(1) | −9(1) |
| C(7B) | 60(1) | 49(1) | 28(1) | −3(1) | −18(1) | −11(1) |
| C(8B) | 34(1) | 32(1) | 29(1) | −6(1) | −7(1) | −8(1) |
| C(9B) | 40(1) | 29(1) | 30(1) | −2(1) | −6(1) | −11(1) |
| C(10B) | 35(1) | 30(1) | 28(1) | −3(1) | −4(1) | −8(1) |
| C(11B) | 58(1) | 28(1) | 31(1) | 0(1) | −10(1) | −9(1) |
| C(12B) | 36(1) | 31(1) | 26(1) | −3(1) | −5(1) | −10(1) |
| C(13B) | 52(1) | 31(1) | 30(1) | −4(1) | −7(1) | −10(1) |
| C(14B) | 54(1) | 34(1) | 32(1) | 1(1) | −10(1) | −10(1) |
| C(15B) | 41(1) | 43(1) | 28(1) | −2(1) | −8(1) | −13(1) |
| C(16B) | 50(1) | 38(1) | 31(1) | −10(1) | −6(1) | −11(1) |
| C(17B) | 49(1) | 30(1) | 31(1) | −4(1) | −7(1) | −11(1) |
| C(18B) | 53(1) | 58(1) | 30(1) | −6(1) | −11(1) | −9(1) |
| O(2B) | 40(1) | 61(1) | 35(1) | 5(1) | −10(1) | −13(1) |
| O(3B) | 47(1) | 56(1) | 35(1) | 7(1) | −15(1) | −19(1) |
| C(19B) | 39(1) | 35(1) | 35(1) | 2(1) | −12(1) | −3(1) |
| C(20B) | 42(1) | 53(1) | 35(1) | 3(1) | −9(1) | −10(1) |
| C(21B) | 39(1) | 46(1) | 36(1) | 1(1) | −9(1) | −2(1) |
| C(22B) | 44(1) | 57(1) | 36(1) | 1(1) | −9(1) | −4(1) |
| C(23B) | 47(1) | 60(1) | 40(1) | −4(1) | −14(1) | 1(1) |
| C(24B) | 66(2) | 80(2) | 40(1) | −11(1) | −19(1) | 9(1) |

TABLE 5
Hydrogen coordinates (×10⁴) and isotropic displacement parameters
(Å² × 10³) for Example 3.

| | x | y | z | U(eq) |
|---|---|---|---|---|
| H(2A) | −2030(20) | 1702(19) | 10598(15) | 50(6) |
| H(1A) | −1640(20) | 2091(19) | 9670(16) | 53(6) |
| H(3A) | 4274(19) | −236(17) | 9173(13) | 38(5) |
| H(4A) | 4120(20) | −721(19) | 10608(14) | 51(6) |
| H(5A) | 2290(20) | −204(19) | 11552(15) | 54(6) |
| H(6A) | 3024(19) | 1241(18) | 6852(13) | 44(6) |
| H(7A) | 2762(18) | −2349(17) | 7728(13) | 42(5) |
| H(8A) | 3752(19) | 1457(18) | 5475(13) | 43(6) |
| H(9A) | 4060(20) | 1834(19) | 4043(14) | 51(6) |
| H(10A) | 3160(20) | −1090(20) | 3812(15) | 58(7) |
| H(11A) | 2874(19) | −1491(19) | 5273(14) | 49(6) |
| H(12A) | 10300(20) | 1610(20) | 8467(17) | 65(8) |
| H(13A) | 8260(20) | 2740(20) | 6989(15) | 55(7) |
| H(14A) | 10470(20) | 1480(20) | 6446(15) | 56(7) |
| H(15A) | 8710(20) | 2300(20) | 5462(15) | 57(7) |
| H(16A) | 10950(30) | 1100(20) | 4980(17) | 73(8) |
| H(18A) | 10520(30) | 1960(30) | 3680(20) | 96(11) |
| H(19A) | 9280(30) | 1890(30) | 4010(20) | 92(11) |
| H(17A) | 10090(40) | 830(40) | 3810(30) | 131(16) |
| H(1B) | 6421(18) | 3524(17) | 5506(14) | 38(5) |
| H(2B) | 6970(20) | 3964(18) | 4633(14) | 46(6) |
| H(3B) | 600(20) | 3979(19) | 5282(14) | 52(6) |
| H(4B) | 1060(20) | 4420(20) | 3828(15) | 56(7) |
| H(5B) | 3025(19) | 4582(18) | 3083(14) | 49(6) |
| H(6B) | 1729(17) | 3233(17) | 7847(12) | 34(5) |
| H(7B) | 1940(19) | 6764(19) | 6838(13) | 46(6) |
| H(8B) | 2148(19) | 2884(19) | 9101(14) | 48(6) |
| H(9B) | 2040(20) | 2560(20) | 10556(16) | 65(7) |
| H(10B) | 1160(20) | 5850(20) | 10770(15) | 58(7) |
| H(11B) | 1203(18) | 6189(18) | 9359(13) | 41(5) |
| H(12B) | 4430(30) | 3030(20) | 6396(19) | 82(9) |
| H(13B) | 4310(20) | 2470(20) | 8450(15) | 63(7) |
| H(14B) | 6230(20) | 3545(19) | 8341(14) | 51(6) |
| H(15B) | 4670(20) | 2810(20) | 9829(15) | 55(7) |
| H(16B) | 6530(30) | 3980(20) | 9668(18) | 78(8) |
| H(18B) | 5280(40) | 4300(40) | 11140(20) | 123(14) |
| H(19B) | 6280(30) | 3390(30) | 11150(20) | 107(12) |
| H(17B) | 5060(30) | 3160(30) | 11150(20) | 109(13) |

TABLE 6
Torsion angles [°] for Example 3.

| | |
|---|---|
| C(3A)—N(2A)—C(1A)—N(1A) | −179.01(15) |
| C(3A)—N(2A)—C(1A)—S(1A) | 2.67(17) |
| C(2A)—S(1A)—C(1A)—N(2A) | −2.78(13) |
| C(2A)—S(1A)—C(1A)—N(1A) | 178.82(14) |
| C(1A)—S(1A)—C(2A)—C(7A) | −176.64(17) |
| C(1A)—S(1A)—C(2A)—C(3A) | 1.94(12) |
| C(1A)—N(2A)—C(3A)—C(4A) | 177.88(15) |
| C(1A)—N(2A)—C(3A)—C(2A) | −1.07(19) |
| C(7A)—C(2A)—C(3A)—N(2A) | 177.74(15) |
| S(1A)—C(2A)—C(3A)—N(2A) | −0.96(17) |
| C(7A)—C(2A)—C(3A)—C(4A) | −1.3(2) |
| S(1A)—C(2A)—C(3A)—C(4A) | 180.00(12) |
| N(2A)—C(3A)—C(4A)—C(5A) | −178.20(16) |
| C(2A)—C(3A)—C(4A)—C(5A) | 0.7(2) |
| N(2A)—C(3A)—C(4A)—O(1A) | 5.7(2) |
| C(2A)—C(3A)—C(4A)—O(1A) | −175.39(13) |
| C(8A)—O(1A)—C(4A)—C(5A) | 75.7(2) |
| C(8A)—O(1A)—C(4A)—C(3A) | −108.20(17) |
| C(3A)—C(4A)—C(5A)—C(6A) | 0.1(3) |
| O(1A)—C(4A)—C(5A)—C(6A) | 176.11(16) |
| C(4A)—C(5A)—C(6A)—C(7A) | −0.4(3) |
| C(3A)—C(2A)—C(7A)—C(6A) | 1.0(3) |
| S(1A)—C(2A)—C(7A)—C(6A) | 179.41(15) |
| C(5A)—C(6A)—C(7A)—C(2A) | −0.1(3) |
| C(11A)—N(3A)—C(8A)—O(1A) | 178.79(16) |
| C(11A)—N(3A)—C(8A)—C(9A) | −1.0(2) |
| C(4A)—O(1A)—C(8A)—N(3A) | 3.4(2) |
| C(4A)—O(1A)—C(8A)—C(9A) | −176.30(14) |
| N(3A)—C(8A)—C(9A)—C(10A) | 0.5(2) |
| O(1A)—C(8A)—C(9A)—C(10A) | −179.28(14) |
| C(11A)—N(4A)—C(10A)—C(9A) | −0.8(3) |
| C(11A)—N(4A)—C(10A)—C(12A) | 178.89(16) |
| C(8A)—C(9A)—C(10A)—N(4A) | 0.5(2) |
| C(8A)—C(9A)—C(10A)—C(12A) | −179.25(15) |
| C(10A)—N(4A)—C(11A)—N(3A) | 0.3(3) |
| C(8A)—N(3A)—C(11A)—N(4A) | 0.6(3) |
| N(4A)—C(10A)—C(12A)—C(13A) | −165.65(17) |
| C(9A)—C(10A)—C(12A)—C(13A) | 14.1(3) |
| N(4A)—C(10A)—C(12A)—C(17A) | 13.3(2) |
| C(9A)—C(10A)—C(12A)—C(17A) | −166.99(17) |
| C(17A)—C(12A)—C(13A)—C(14A) | 1.1(3) |
| C(10A)—C(12A)—C(13A)—C(14A) | 179.98(17) |
| C(12A)—C(13A)—C(14A)—C(15A) | −0.3(3) |
| C(13A)—C(14A)—C(15A)—C(16A) | −0.4(3) |
| C(13A)—C(14A)—C(15A)—C(18A) | 177.39(17) |
| C(14A)—C(15A)—C(16A)—C(17A) | 0.3(3) |
| C(18A)—C(15A)—C(16A)—C(17A) | −177.48(18) |
| C(15A)—C(16A)—C(17A)—C(12A) | 0.4(3) |
| C(13A)—C(12A)—C(17A)—C(16A) | −1.1(3) |
| C(10A)—C(12A)—C(17A)—C(16A) | 179.94(17) |
| C(14A)—C(15A)—C(18A)—F(1A) | −99.5(2) |
| C(16A)—C(15A)—C(18A)—F(1A) | 78.3(2) |
| C(14A)—C(15A)—C(18A)—F(2A) | 21.2(3) |
| C(16A)—C(15A)—C(18A)—F(2A) | −161.05(18) |
| C(14A)—C(15A)—C(18A)—F(3A) | 140.76(19) |
| C(16A)—C(15A)—C(18A)—F(3A) | −41.4(2) |
| O(2A)—C(19A)—C(20A)—C(21A) | −176.5(2) |
| O(3A)—C(19A)—C(20A)—C(21A) | 3.0(3) |
| C(19A)—C(20A)—C(21A)—C(22A) | 178.11(19) |
| C(20A)—C(21A)—C(22A)—C(23A) | 179.6(2) |
| C(21A)—C(22A)—C(23A)—C(24A) | 180.0(3) |
| C(3B)—N(2B)—C(1B)—N(1B) | 177.74(15) |
| C(3B)—N(2B)—C(1B)—S(1B) | −2.69(17) |
| C(2B)—S(1B)—C(1B)—N(2B) | 2.02(13) |
| C(2B)—S(1B)—C(1B)—N(1B) | −178.39(14) |
| C(1B)—S(1B)—C(2B)—C(7B) | 177.83(18) |
| C(1B)—S(1B)—C(2B)—C(3B) | −0.70(12) |
| C(1B)—N(2B)—C(3B)—C(4B) | −174.93(16) |
| C(1B)—N(2B)—C(3B)—C(2B) | 2.13(19) |
| C(7B)—C(2B)—C(3B)—N(2B) | −179.29(16) |
| S(1B)—C(2B)—C(3B)—N(2B) | −0.64(18) |
| C(7B)—C(2B)—C(3B)—C(4B) | −2.0(2) |
| S(1B)—C(2B)—C(3B)—C(4B) | 176.68(12) |
| N(2B)—C(3B)—C(4B)—C(5B) | −179.87(16) |
| C(2B)—C(3B)—C(4B)—C(5B) | 3.1(2) |
| N(2B)—C(3B)—C(4B)—O(1B) | −2.2(2) |
| C(2B)—C(3B)—C(4B)—O(1B) | −179.19(14) |
| C(8B)—O(1B)—C(4B)—C(5B) | −99.71(19) |
| C(8B)—O(1B)—C(4B)—C(3B) | 82.57(19) |
| C(3B)—C(4B)—C(5B)—C(6B) | −1.6(3) |
| O(1B)—C(4B)—C(5B)—C(6B) | −179.26(17) |
| C(4B)—C(5B)—C(6B)—C(7B) | −1.2(3) |
| C(5B)—C(6B)—C(7B)—C(2B) | 2.3(3) |
| C(3B)—C(2B)—C(7B)—C(6B) | −0.7(3) |
| S(1B)—C(2B)—C(7B)—C(6B) | −179.06(16) |
| C(11B)—N(3B)—C(8B)—O(1B) | −179.40(16) |
| C(11B)—N(3B)—C(8B)—C(9B) | 0.5(3) |
| C(4B)—O(1B)—C(8B)—N(3B) | 5.5(2) |
| C(4B)—O(1B)—C(8B)—C(9B) | −174.41(15) |
| N(3B)—C(8B)—C(9B)—C(10B) | −2.3(3) |
| O(1B)—C(8B)—C(9B)—C(10B) | 177.62(15) |
| C(11B)—N(4B)—C(10B)—C(9B) | −0.5(3) |
| C(11B)—N(4B)—C(10B)—C(12B) | 178.36(16) |
| C(8B)—C(9B)—C(10B)—N(4B) | 2.2(3) |
| C(8B)—C(9B)—C(10B)—C(12B) | −176.56(15) |
| C(10B)—N(4B)—C(11B)—N(3B) | −1.5(3) |
| C(8B)—N(3B)—C(11B)—N(4B) | 1.6(3) |
| N(4B)—C(10B)—C(12B)—C(17B) | 15.3(2) |
| C(9B)—C(10B)—C(12B)—C(17B) | −165.86(17) |
| N(4B)—C(10B)—C(12B)—C(13B) | −163.88(17) |
| C(9B)—C(10B)—C(12B)—C(13B) | 15.0(3) |
| C(17B)—C(12B)—C(13B)—C(14B) | −0.9(3) |
| C(10B)—C(12B)—C(13B)—C(14B) | 178.24(17) |
| C(12B)—C(13B)—C(14B)—C(15B) | 0.4(3) |
| C(13B)—C(14B)—C(15B)—C(16B) | 0.4(3) |
| C(13B)—C(14B)—C(15B)—C(18B) | −177.02(18) |
| C(14B)—C(15B)—C(16B)—C(17B) | −0.6(3) |
| C(18B)—C(15B)—C(16B)—C(17B) | 176.82(18) |
| C(15B)—C(16B)—C(17B)—C(12B) | 0.0(3) |
| C(13B)—C(12B)—C(17B)—C(16B) | 0.8(3) |
| C(10B)—C(12B)—C(17B)—C(16B) | −178.42(16) |
| C(16B)—C(15B)—C(18B)—F(1B) | −103.6(3) |
| C(14B)—C(15B)—C(18B)—F(1B) | 73.8(3) |
| C(16B)—C(15B)—C(18B)—F(3B) | 20.9(3) |
| C(14B)—C(15B)—C(18B)—F(3B) | −161.7(2) |
| C(16B)—C(15B)—C(18B)—F(2B) | 138.3(2) |
| C(14B)—C(15B)—C(18B)—F(2B) | −44.3(3) |
| O(2B)—C(19B)—C(20B)—C(21B) | 12.8(3) |
| O(3B)—C(19B)—C(20B)—C(21B) | −166.10(19) |
| C(19B)—C(20B)—C(21B)—C(22B) | 175.03(19) |
| C(20B)—C(21B)—C(22B)—C(23B) | −175.2(2) |
| C(21B)—C(22B)—C(23B)—C(24B) | 178.6(2) |

TABLE 7
Hydrogen bonds for Example 3 [Å and °].

| D-H . . . A | d(D-H) | d(H . . . A) | d(D . . . A) | <(DHA) |
|---|---|---|---|---|
| N(1A)—H(2A) . . . N(3A)#1 | 0.86(2) | 2.24(2) | 3.035(2) | 154(2) |
| N(1A)—H(1A) . . . O(2A)#2 | 0.93(2) | 1.99(2) | 2.897(2) | 164(2) |
| O(3A)—H(12A) . . . N(2A)#3 | 0.87(3) | 1.85(3) | 2.723(2) | 176(3) |
| N(1B)—H(1B) . . . O(2B) | 0.86(2) | 2.11(2) | 2.959(2) | 169.4(19) |
| N(1B)—H(2B) . . . N(3B)#4 | 0.91(2) | 2.14(2) | 3.021(2) | 162(2) |
| O(3B)—H(12B) . . . N(2B) | 0.92(3) | 1.77(3) | 2.6865(19) | 174(3) |

### EXAMPLE 4

Single crystal structure of the N-(4-(6-(4-(trifluoromethyl)phenyl)pyrimidin-4-yloxy)benzo[d]thiazol-2-yl)acetamide freebase (Example 4) was determined on a Rigaku AFC7R diffractometer with graphite monochromated Cu-Ka radiation. Data was collected at 20° C., to a maximum 2Θ value of 120.1°.

### EXAMPLE 5

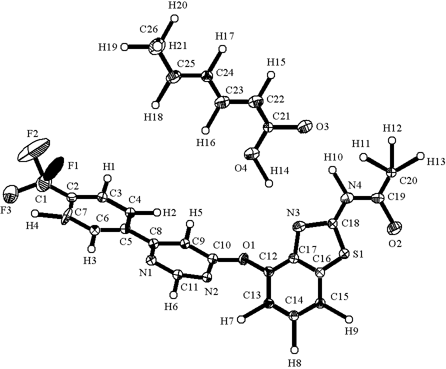

The single crystal structure of N-(4-(6-(4-(trifluoromethyl)phenyl)pyrimidin-4-yloxy)benzo[d]thiazol-2-yl)acetamide sorbic acid co-crystal (Example 5) was determined on a Rigaku FR-E SuperBright rotating copper anode generator equipped with a Rigaku Saturn 92 CCD area detector, AFC11 goniostat, and the Varimax optics. Data was collected at −160° C., to a maximum 2Θ value of 108.5°, refined to 0.95 Å, and processed using CrystalClear (Rigaku). Both structures were solved by direct methods and expanded using Fourier techniques. The position of the hydrogen bonds was determined using the Mercury 1.4 software using standard settings.

### Biological Studies

### Pharmacokinetic Parameters and Summary Statistics for Male Cynomolgus Monkeys Following Nasogastric Gavage (2% Pluronic F108 in OraPlus Suspension) or Oral (Tablet Formulations) Administration (N=4/group)

| | | Tₘₐₓ | Cₘₐₓ | AUC_{0-inf} (ng- | Fᵣₑₗ |
|---|---|---|---|---|---|
| Formulation | | (hr)^{a} | (ng/mL) | hr/mL) | (%) |
| 2% Pluronic | Mean | 3 | 2210 | 102000 | |
| F108 in OraPlus | SD | 1.0-4.0 | 277 | 20000 | |
| Suspension | % CV | | 13 | 20 | |
| Example 4 | Mean | 8 | 379 | 19200 | 18.8 |
| Form A Tablet | SD | 8.0-12.0 | 20.3 | 3160 | 3.09 |
| | % CV | | 5.3 | 17 | 17 |
| Example 5, | Mean | 1.5 | 1400 | 52700 | 51.6 |
| Physical Blend | SD | 1.0-8.0 | 673 | 30800 | 30.1 |
| | % CV | | 48 | 58 | 58 |
| Example 5, | Mean | 5 | 1480 | 65500 | 64.1 |
| Fluid Bed | SD | 2.0-12.0 | 658 | 19700 | 19.3 |
| Granulation | % CV | | 45 | 30 | 30 |

^{a}Presented as median and range.
Tₘₐₓ = Time at which Cmax was observed
Cₘₐₓ = Maximum observed plasma concentration
AUC_{0-inf} = Area under the plasma concentration-time curve from time zero to infinity
Fᵣₑₗ = Relative bioavailability, calculated by: Individual AUC_{0-inf} Tablet/Mean AUC_{0-inf} Suspension

Oral administration of the Example 4 in tablet form yielded mean Cₘₐₓ and AUC values approximately 17-19% those of the suspension formulation of Example 4, with relatively low inter-animal variability in exposure (% CV 5-17). Oral administration of the Example 5 “in situ” sorbic acid cocrystal/physical blend tablet yielded mean Cₘₐₓ and AUC values approximately 52-63% those of the suspension formulation, with higher inter-animal variability in exposure (% CV˜50-60). Oral administration of the Example 5 “in situ” sorbic acid cocrystal/physical blend tablet yielded mean Cₘₐₓ and AUC values approximately 65% those of the suspension formulation, with comparable or somewhat lower inter-animal variability in exposure (% CV˜30-45) relative to the “in situ” sorbic acid co-crystal formulation.

The foregoing is merely illustrative of the invention and is not intended to limit the invention to the disclosed compounds. Variations and changes, which are obvious to one skilled in the art, are intended to be within the scope and nature of the invention, which are defined, in the appended claims.

From the foregoing description, one skilled in the art can easily ascertain the essential characteristics of this invention, and without departing from the spirit and scope thereof, can make various changes and modifications of the invention to adapt it to various usages and conditions.

We claim:

1. A pharmaceutical co-crystal comprising:
an active pharmaceutical ingredient; and
a co-crystal agent having the structure R¹—C(═O)XH, wherein X is O, N(C₁₋₆alkyl) or NH and R¹ is a C₃₋₈alkyl group containing at least one trans-oriented double bond and being substituted by 0, 1, 2, 3 or 4 groups independently selected from halo, phenyl and hydroxyl.

2. A pharmaceutical co-crystal according to claim 1, wherein the co-crystal agent is selected from sorbic acid, trans-2-hexenoic acid, trans-3-hexenoic acid, trans-4-hexenoic acid, trans-2-butenoic acid, trans-2-pentenoic acid, trans-3-pentenoic acid, trans-2,4-pentadienoic acid.

3. A pharmaceutical co-crystal according to claim 1, wherein the co-crystal agent is sorbic acid.

4. A method of manufacturing a pharmaceutical co-crystal according to claim 1, comprising the steps of:
contacting a co-crystal agent with an active pharmaceutical ingredient;
isolating the formed pharmaceutical co-crystal.

5. A method according to claim 4, wherein the contacting occurs with both the co-crystal agent and the active pharmaceutical ingredient dissolved in a solvent.

6. A method according to claim 4, wherein the contacting occurs in a milling device with both the co-crystal agent and the active pharmaceutical ingredient being solids.

7. A method for increasing the bioavailability of an active pharmaceutical ingredient in a mammal comprising the steps of contacting the active pharmaceutical ingredient with a co-crystal agent; and forming a co-crystal comprising the active pharmaceutical ingredient and the co-crystal agent.

8. A pharmaceutical composition comprising:
a co-crystal according to claim 1; and
a pharmaceutically-acceptable carrier or diluent.
